(19) United States
(12) Patent Application Publication
(10) Pub. No.: US 2017/0156296 A1
(43) Pub. Date: Jun. 8, 2017

(54) Device for positioning the head of an animal and method of use thereof

(71) Applicant: George Michael Hayman, JR., Pooler, GA (US)

(72) Inventor: George Michael Hayman, JR., Pooler, GA (US)

(21) Appl. No.: 15/238,712

(22) Filed: Aug. 16, 2016

Related U.S. Application Data
(60) Provisional application No. 62/205,736, filed on Aug. 16, 2015.

(51) Int. Cl. A01K 29/00; A61D 3/00; A61B 5/0478; A61B 5/00; A01K 15/04; A01K 15/02

(52) U.S. Cl. CPC A01K 29/005; A01K 15/04; A01K 15/02; A61B 5/0478; A61B 5/702; A61D 3/00; A61B 2503/40; A61D 2003/003

(57) ABSTRACT

A device, system, or method to aid in conducting brain activity analysis on an animal. The device may include a headrest for receiving at least part of the head of the animal, and a tube connected to the headrest for passing a treat to the animal through the tube so the animal is trained to position his or her head on the headrest and to maintain his or her head on the headrest for the time it takes to carry out the brain activity analysis. The animal is trained by the treat being passed to the animal through the tube. The device may be used with a replica to further train the animal prior to the device being used with a machine that may carry out brain activity analysis. The device may allow for the presence of stimuli and distractions as part of the training of the animal.

### CROSS-REFERENCE TO RELATED APPLICATION

This application claims priority to and the benefit of the prior filed co-pending and commonly owned Provisional Application filed in the United States Patent and Trademark Office (USPTO) on Aug. 16, 2015, assigned Ser. No. 62/205,736, and entitled A Device for Positioning a Head of an Animal to Allow for Brain Activity Analysis and Methods for Use Thereof which is incorporated herein by reference in its entirety at least to provide continuity of disclosure.

### FIELD OF THE INVENTION

The field of the invention relates to devices and apparatuses used in brain activity analysis, and particularly, relates to devices and apparatuses used for positioning the head of an animal for brain activity analysis.

### BACKGROUND

Brain activity analysis on an animal may yield useful results. The brain activity analysis may yield various types of information. This information may include whether a particular animal may be aggressive, passive, a good candidate for a sporting dog, a good candidate for a working dog such as a police dog, drug enforcement dog, customs dog, military dog, search and rescue dog, cadaver dog, and/or other information.

The information obtained from brain activity analysis may be useful to many different fields of endeavor. For example, a breeder may use the information to breed an animal with desired characteristics. As another example, a trainer may use the information to determine if a particular dog is a good candidate for a particular type of training. Also, a zookeeper may use the information to care for a particular animal. In addition, a person may use the information to determine whether a specific animal is able or better able than other animals to carry out a specific task.

### SUMMARY

Advantageously, the invention may relate to a device, system, and/or method to aid in conducting brain activity analysis on an animal.

A device according to the invention may include a headrest for receiving at least part of the head of the animal. The headrest may be connected by a neck to a tube. The headrest, neck, and tube may be of all one piece, i.e., integrally made. The tube may be connected to the headrest for passing a treat to the animal through the tube so the animal may be trained to position his or her head on the headrest and to maintain his or her head on the headrest for the time it takes to carry out brain activity analysis.

A system according to the invention may aid in conducting brain activity analysis on an animal. The system may include a device positioned within a machine that carries out brain activity analysis. The device may include a headrest for receiving at least part of the head of the animal. The device may include a tube connected to the headrest for passing a treat to the animal through the tube so the animal is trained to position his or her head on the headrest and to maintain his or her head on the headrest for the time it takes to carry out the brain activity analysis by the machine.

The system may optionally include a replica of the machine. The device may be positionable within the replica for training the animal to position his or her head on the device within the replica prior to training the animal to position his or head on the device within the BAA machine. The device may be further positionable within the machine after the animal is trained to position his or head on the device within the replica so that the animal is trainable to position his or her head on the device within the machine,

The invention may include a method to conduct brain activity analysis (BAA) using a machine on an the animal. The method may include training an animal to position his or her head on a headrest of a cup positioned within the BAA machine so that the animal maintains the position of his or her head on the headrest of the cup for the time it takes for the machine to carry out the BAA on the animal. The method also may include conducting the brain activity analysis using the machine with the animal positioned on the device embodiment within the machine. Additionally, the method may provide that prior to training the animal to position his or head on the headrest of the cup positioned within the BAA machine, the animal may be trained to position his or head on the headrest of the cup before it is positioned within the BAA machine. Further, the method may provide that after the animal is trained to position his or head on the headrest of the cup before the cup is positioned within the BAA machine, and prior to training the animal to position his or head on the headrest of the cup within the BAA machine, the animal may be trained to position his or head on the headrest of the cup positioned in a replica of the machine.

The method also may including that after training the animal to position his or head on the headrest of the cup positioned in the replica of the machine, the animal may be trained to position his or head on the headrest of the cup positioned in the replica the presence of one or more stimuli or distractions.

The method further may provide that after conducting the brain activity analysis using the machine with the animal positioned on the device embodiment within the machine, the brain activity analysis may be conducted using the machine with the animal positioned on the device embodiment within the machine in the presence of one or more stimuli or distractions.

Exemplary embodiments of the invention are described herein in order to enable those skilled in the art to utilize the inventions including various embodiments and various modifications as are suited to the particular uses contemplated.

### BRIEF DESCRIPTION OF THE DRAWINGS

FIG. 1 depicts a possible exemplary use of an embodiment of the invention with a magnetic resonance imaging machine and a dog.

FIG. 2 is a perspective view of an exemplary embodiment of the invention.

FIG. 3 illustrates a possible exemplary use of an embodiment of the invention with an apparatus constructed to resemble a magnetic resonance imaging machine.

FIG. 4 shows a possible exemplary use of an embodiment of the invention with an apparatus constructed to resemble a magnetic resonance imaging machine.

FIG. 5 is a flow diagram illustrating an exemplary embodiment of the invention.

### DETAILED DESCRIPTION

The invention is described herein with reference to exemplary embodiments, alternative embodiments, attached drawings, and other accompanying materials. The invention, however, may be embodied in many different forms carried out in a variety of ways, and should not be construed as limited to the embodiments and other materials set forth herein or accompanying herewith. The exemplary embodiments that are described and shown herein are only a few examples of ways to implement the invention. There may be other ways. The subject matter herein is described as “the invention,” but the subject matter may include one or more inventions.

Herein, in terms of nomenclature, the singular includes the plural, the masculine includes the feminine or neutral, and a person includes a partnership, association, company and/or corporation (and/or the like). An embodiment, in whole or in part, may be an apparatus, a device, a kit, a method, and/or a system (and/or the like), unless otherwise noted. A user of an embodiment of the invention also may be referred to herein as a “trainer,” and/or “handler.” Moreover, like numerals indicate like elements throughout the figures.

The invention relates to a device for positioning the head of an animal. The device may allow for activity analysis of the brain of the animal. The invention, however, should not be limited to this purpose. The device may be used for other purposes. Brain activity analysis (BAA) is used herein as an example of a use of the device, but readers will understand the device may be used for other purposes. Further, the device may be used in or with one or more apparatuses and/or one or more methods.

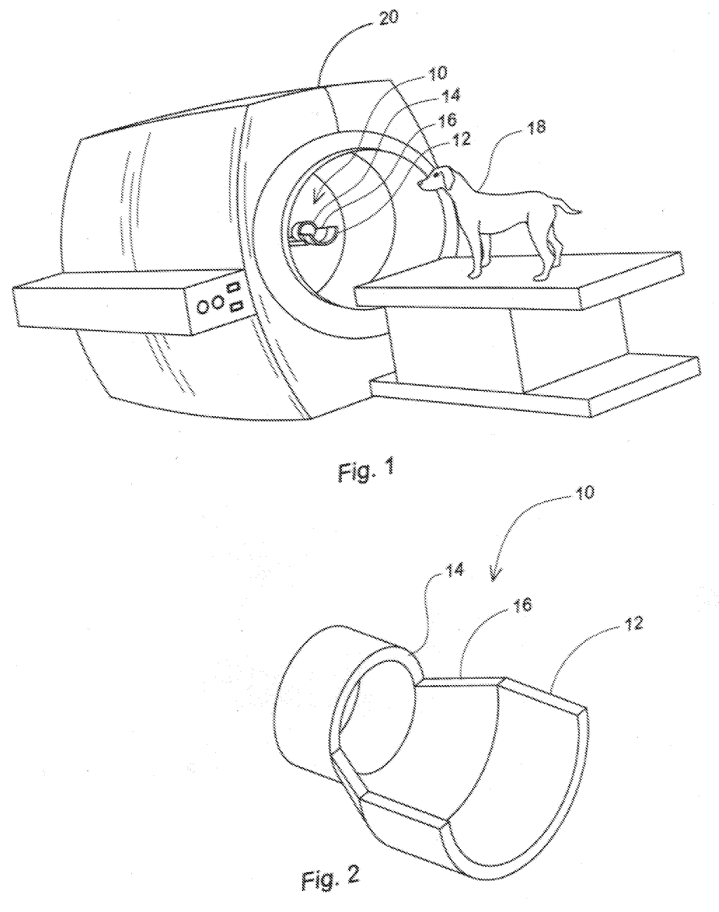

FIG. 1 depicts an exemplary embodiment of the invention 10 in use with an apparatus that may be used in brain activity analysis of an animal such as a dog. In this embodiment, the device 10 includes a headrest 12 connected to a tube 14. In this embodiment, the device 10 includes a neck 16 that connects the headrest 12 to the tube 14, but other embodiments may vary. Generally stated, the head of an animal such as the dog 18 shown in FIG. 1 may be positioned to rest on the headrest 12 without further restraint typically. The positioning of the head may be accomplished by training of the animal and/or otherwise.

A brain activity analysis (BAA) may be carried out by an apparatus 20 with respect to the animal 18 whose head is resting on the headrest 12 of the device 10 positioned as part of the apparatus 20. The device 10 may be used with the animal 18 to maintain the position of his or her head for a period of time on the headrest 12. Typically, the period of time the head of the animal 18 may be positioned on the headrest 12 may be sufficient to allow for the brain activity analysis (BAA) to be carried out in whole and/or in part. The device 10 may be used to train the animal 18 to remain in position on the device 10 for the brain activity analysis (BAA), for example, by offering the animal 18 one or more treats through the tube 14 of the device 10, and/or otherwise. In use, the device 10 may be used to provide the animal 18 with treats through the tube 14 to maintain his or her position during brain activity analysis (BAA).

The general configurations of each of the headrest 12, tube 14, and neck 16 of the exemplary device 10 are now described.

The headrest 12 in this exemplary embodiment 10 is a bottom half of a generally round cylinder. From the top of the right side to the top of the left side, the headrest 12 is about 4 inches across, which is the diameter of the cylinder of which the headrest 12 is the bottom half. The headrest 12 is about 1.5 inches in depth from the front of the headrest 12 to where it connects with the neck 16 of the device. The thickness of the headrest 12 is about 1/32nd of an inch. Other embodiments may vary.

The tube 14 of the exemplary device 10 is a round cylinder with a diameter of about 2.75 inches, a depth of about 1.5 inches, and a thickness of about 1/32nd of an inch. Other embodiments may vary.

In the exemplary embodiment 10, the headrest 12 of the device 10 is connected to the tube 14 by the neck 16. Other embodiments may fail to include a neck and/or vary in other ways. The neck 16 is shaped like half of a cone that decreases in diameter from its largest diameter at about 4 inches at its connection to the headrest 12 to its smallest diameter at about 2 inches at its connection to the tube 14. Other embodiments may vary.

The exemplary device 10 is integrally made or unitary. In other words, all of its elements (headrest 12, tube 14, and neck 16) are made of one piece. In this example, the device 10 is made of polyvinyl chloride (PVC). Other embodiments may vary. More particularly, the device 10 may be made from a 2-inch-to-4-inch PVC pipe fitting or coupling such as may be used in plumbing, but with the appropriate part(s) of the PVC pipe fitting cut away to render the device 10.

An animal such as a dog 18 may be trained to the device 10 in its use with a brain activity analysis machine such as and/or including a magnetic resonance imagining (MM) machine, and/or other machine. A machine also may be referred to herein as “apparatus.” The animal 18 may first be to trained or conditioned to just the device 10. By “just the device 10,” it is meant that an animal 18 may be trained in all respects regarding the device 10 without distractions and/or stimuli, without use of the device 10 with other elements, and without reference to an MM and/or other machine and/or system. An animal 18 also may be trained or conditioned to the device 10 (or similar embodiment) through the use of a replica of a machine that may be used for brain activity analysis (BAA). An animal 18 further may be trained or conditioned to the device 10 (or similar embodiment) as it may be used with a machine that carries out brain activity analysis (BAA). Once trained, an animal 18 may have its brain activity analyzed through use of the device 10 (or similar embodiment) with a machine that carries out BAA.

FIG. 2 shows an exemplary device 10 as may be used to condition an animal 18 to “just the device 10.” A trainer or handler may hold the device 10 to familiarize the animal 18 to the device 10. Further, the trainer may train the animal 18 to put his or her head on the device 10 while the trainer holds the device 10. Still while holding the device 10, the trainer may train the animal 18 to hold his or her head without movement on the device 10 for longer and longer periods until the trainer is satisfied. At some point, the trainer may relinquish holding the device 10, but instead use a prop to hold the device 10, and then the trainer may condition the animal 18 to put his or her head on the device 10 and hold the position without movement for longer and longer periods until the trainer is satisfied.

When reference is made herein to an animal “putting his or her head on or in a device according to the invention”, such reference may include the animal putting less than all of his or her head on or in a device according to the invention. For example, an animal such as a dog may put his or her muzzle, and in particular, the bottom of his or her muzzle on the device 10, and particularly, on the headrest 12 of the device 10.

After being conditioned to just the device 10 (with and/or without the trainer holding the device 10), the animal 18 may be conditioned to the device 10 with one or more distractions and/or stimuli such as sound, smell, etc. For example, the animal 18 may be provided with one or more stimuli, distractions, etc. while the animal 18 is in position on the device 10. The use of one or more other elements (such as a tube, an oxygen mask, a stick) for delivering such stimuli and/or distractions (and/or for other reason(s)) in connection with the device 10 (or similar embodiment) is discussed in further detail below.

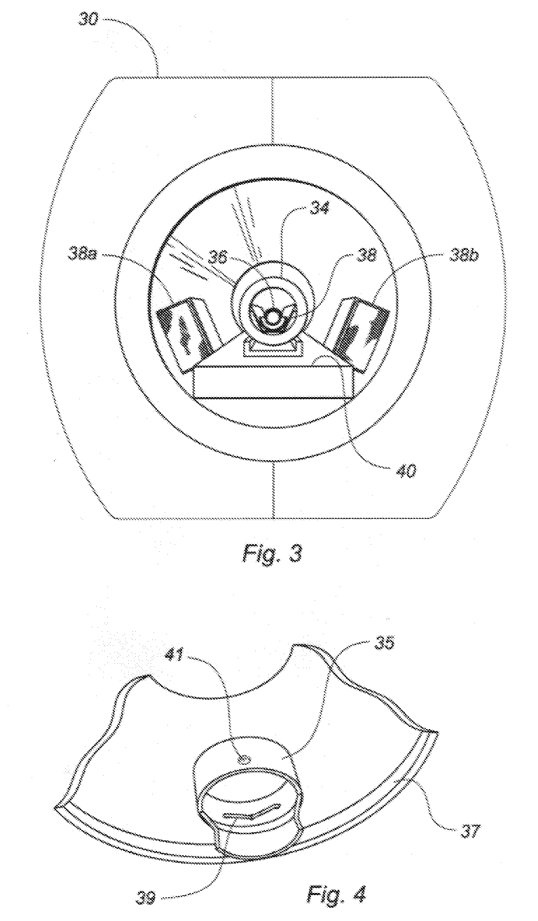

After such conditioning as discussed in the previous paragraph, (or without such conditioning), the animal 18 may be trained to a replica of the machine or apparatus with which the device 10 is to be used. The invention includes an embodiment including a device 10 (or similar device) used with a replica 30 of a machine that may be used for brain activity analysis (BAA) (or something similar to replica 30). FIG. 3 shows an apparatus 30 built to resemble a machine that may be used for brain activity analysis (BAA). The replica 30 includes a relatively large cylinder 32 large enough to resemble the overall size of a machine used for brain activity analysis (BAA). Within the large cylinder 32, FIG. 3 shows a smaller cylinder 34 containing an exemplary embodiment 36 of the invention. The exemplary embodiment 36 compares to the exemplary device 10 at least in that the exemplary embodiment 36 allows the animal to position his or her head within the exemplary embodiment 36 and maintain such position without movement such as would be desirable for brain activity analysis (BAA).

The relatively large cylinder 32 (also referred to as a “chamber”) of the replica 30 may be described generally as a tubular or round cylindrical “tub” lying on its side so the large opening of the tub is positioned substantially vertically on one side of the chamber, and the smaller opening of the tub is also positioned substantially vertically, but opposite to and generally parallel to the larger opening. The “tub” may resemble part of a magnetic resonance imaging (MRI) system.

The exemplary embodiment 36 is positioned substantially inside the tub, and particularly, in or near the bottom of the large opening of the tub and near the edge of the large opening. This embodiment of the exemplary embodiment 36 has a headrest that is curved so that a cross section of the headrest forms a half circle. The curve of the headrest advantageously may allow for relatively easy positioning of the muzzle of the animal on the headrest and/or may allow for relatively easy positioning of the headrest in the arc of the bottom tubular wall of the tub (in another embodiment).

An animal may place his or her head on the headrest of the exemplary embodiment 36 within the replica 30. Generally, the bottom of the animal's chin rests on the headrest. The exemplary embodiment 36 may be positioned in the tub so that placement of the animal's head on the headrest of the exemplary embodiment 36 positions the animal's head substantially within the tub. This positioning tries to substantially duplicate and/or mimic what would be the positioning of the animal's head when the animal is within a machine that carries out brain activity analysis (BAA).

As noted previously, the animal may be conditioned to the exemplary embodiment 36 prior to the animal being conditioned to the exemplary embodiment as it is positioned in the replica machine 30. An animal may be conditioned to keep his or her head in the appropriate position for sufficient time so that when it comes time for the brain activity analysis, it may be successfully carried out. Once the animal is conditioned sufficiently, the brain activity analysis may be carried out with the animal positioning his or her head on an exemplary embodiment of the invention (device 10, exemplary embodiment 36, or something similar) as it may be used with a machine that carries out brain activity analysis.

As shown in FIG. 3, the exemplary embodiment 36 is held in position within the smaller cylinder 34 by a support 38. The embodiment 36 (and those like it) may be held in position (releasably or not) in the replica 30 and/or in the apparatus for brain activity analysis (BAA) in any appropriate manner. The smaller cylinder 34 may be positioned on a platform 40 within the larger cylinder 32 (again in any appropriate manner) (releasably or not).

FIG. 4 shows an alternative embodiment of the invention 35 as may be used with an alternative replica 37 of a machine that may be used for brain activity analysis (BAA). In some embodiments, alternative embodiment of the invention 35 may be used with a machine that conducts brain activity analysis (BAA). The alternative embodiment 35 is releasably fixed to the alternative replica 37 by a tie 39 that is passed through a hole in the bottom of the alternative embodiment 35 to connect it to the alternative replica 37. Other embodiments may vary, of course.

Referring again to FIG. 3, the platform 40 of the replica 30 may allow an animal to position himself or herself (and/or with the aid of a trainer or handler) within the large cylinder 32 of the replica 30 so the animal may place his or her head on the exemplary embodiment 36 within the smaller cylinder 34 and hold that position without movement.

Rather than being conditioned to the replica 30 with the exemplary embodiment 36 in place, an alternative embodiment of the invention may provide that the animal be conditioned to the replica 30 without the exemplary embodiment 36 in place. By way of further examples, an animal may be conditioned first to either the replica 30 or to the exemplary embodiment 36, and/or the combination thereof.

Advantageously, replica 30 allows for the animal to be conditioned to the replica 30 (with or without the exemplary embodiment 36 or in combination therewith) with one or more distractions and/or stimuli such as sound, smell, etc. For example, the animal may be provided with one or more stimuli, distractions, etc. while the animal is in proximity of the replica 30 and/or the exemplary embodiment 36 and/or the combination. As a further example, the animal may be provided with the one or more stimuli or distractions while in position on the exemplary embodiment 36 as used with the replica 30. FIG. 3 shows a pair of speakers 38a, 38b. An animal may be conditioned to position his or her head on the exemplary embodiment 36 in the replica 30 and to maintain that position without movement even in the presence of sound stimuli from one or both of the speakers 38a, 38b.

The stimuli and distractions may be provided to the animal through one or more apparatuses that may be used in conjunction with the device 10 and/or the exemplary embodiment 36 as the device 10 or exemplary embodiment 36 may be used with a replica (such as replica 30) and/or a machine or apparatus that may carry out the brain activity analysis (BAA).

For training purposes, an animal may be trained to place his or her on a device according to the invention (within a replica such as replica 30 or not) and maintain his or her head in position without movement in the presence of one or more stimuli and/or distractions. For example, a stimuli tube may be inserted into one or more of the elements of a device according to the invention to deliver a stimulus or stimuli to the animal while he or she is in position on the device. FIG. 4 shows a hole 41 in the top of alternative embodiment 35 through which a tube (and/or the like) may be inserted into the interior of the alternative embodiment 35 to deliver one or more stimuli. Other or additional examples of delivering one or more stimulus and/or distractions to an animal with his or her in position in or on an exemplary embodiment of the invention are possible such as a wick (within or outside the exemplary embodiment) that may be dangled just past the animal's nose with a scent and/or other distraction on it.

As another example, an apparatus including a bag such as an oxygen supply bag may be used with an exemplary embodiment such as device 10 or 36 as it is used with a replica and/or of a machine carrying out brain activity analysis (BAA). The oxygen supply bag may deliver odor (and/or other) stimuli to the animal. The animal may require training to be conditioned to the use of the oxygen supply bag prior to use of the oxygen supply bag with an exemplary embodiment of the invention such as device 10 with or without its position in a replica of and/or of a machine carrying out brain activity analysis (BAA).

Yet another example is that the device 10 and/or an alternative embodiment of the invention may be used with a stick (and/or similar device) as part of the method and apparatus to train an animal so the animal maintains its head in such a manner that brain activity analysis (and/or other processes) may be substantially successfully carried out. The stick may carry one or more stimuli, and/or be used for training the positioning the muzzle of the animal on the device 10. The stick may be inserted into the device 10 through a hole (such as previously mentioned hole 41 in embodiment 35), or may be positioned in front of the animal's muzzle on the far side of the device 10 opposite to the insertion side of the device 10. More than one stick may be used in which case, the sticks may be used inside and/or outside of the device 10.

As noted previously, an animal may be trained to position his or her head on an exemplary embodiment of the invention (such as device 10, exemplary embodiment 36, and/or alternative embodiment 37, hereafter “device embodiments”) and to hold that position by a trainer. Also, an animal may be trained to a device embodiment in the presence of stimuli and distractions that may be presented to the animal by one or more elements used with a device embodiment and/or otherwise. Further, an animal may be trained to a device embodiment positioned within a replica of a machine that carries out brain activity analysis (BAA). The training of an animal to a device embodiment within a replica may additionally include training of the animal in the presence of stimuli and/or that may be presented to the animal by one or more elements used with a device embodiment and/or otherwise as it is positioned within the replica.

The invention includes an embodiment including a device embodiment (device 10, exemplary embodiment 36, alternative embodiment 35, or similar device) used with a machine that may be used for brain activity analysis (BAA) (or something similar). In addition, the invention contemplates the training of an animal to a device embodiment within a machine that may carry out brain activity analysis (BAA). Additionally, the invention may allow for the training of the animal in the presence of stimuli and/or that may be presented to the animal by one or more elements used with a device embodiment and/or otherwise as it is positioned within a machine that may carry out brain wave activity. The description above of exemplary device embodiments in use with a replica of a machine that may carry out brain activity analysis (BAA) applies to the use of the exemplary device embodiments with a machine that may carry out brain activity analysis (BAA) (or similar machines in which an animal must position his or her head and keep its position without movement for the time it takes for an activity to take place).

### An Exemplary Method of Use of a Device Embodiment

A device embodiment may be referred to as a K-9 B.A.A. (Brain Activity Analysis) Cup or “cup”. An animal may be trained to position his or her head in the device embodiment properly and long enough so that brain activity analysis or other activity or activities may be carried out.

The training of the animal may include one or more of the following actions:

1. The animal may be familiarized with the device embodiment by a handler and/or other person. For example, the animal may be fed through the tube 14 when the animal's head is positioned on the headrest 12 of the device 10. The feeding may be continuous and consistent for a period of 20-30 seconds. The feeding may be repeated as part of a familiarization process. It may be advisable that the handler shape the animal into a “down” by holding food in the device 10 in front of the animal's nose. If the animal does not go into a “down”, the animal may be taught to go “down,” but away from the device 10.

2. It may not be advisable to allow the animal to leave the device 10 and come back. If the animal leaves the device 10, the handler may return the animal to a place other than the device 10 as appropriate. For animals that may have a “panting issue” such as dogs, the animal may be kenneled prior to training in an effort to reduce the panting problem.

3. After an animal is familiar with the device 10 through action 1 and/or otherwise, it may be advisable to feed the animal in five second intervals increasing interval time gradually to five minutes and/or other specified time.

4. After action 3, the handler may increase the time between feedings. The time may be increased gradually until the increases last between 30 seconds or one minute. Afterwards, increases may be made by larger intervals.

5. As the animal gets more experienced with the device 10 and stays in the device 10, the handler may start randomizing rewards. Also, the handler may start to randomize the amount of time the animal is in the device 10. It may be inadvisable to work for more than 10-15 minutes unless the animal is being scanned or an MM machine is being used.

The above actions may be carried out using only the device 10 and the animal. As noted, however, the device 10 may be used with brain activity analysis that includes a system such as a magnetic resonance imaging (Mill) machine. The animal may be conditioned or socialized to the device 10 as it is used in such a system. The following actions may be followed:

A. The handler may socialize the animal to the machine. To do so, the handler may place food around the room in which the machine is used. The animal may be allowed to find this food in the room by himself or herself. The animal may be allowed to explore the environment of the room.

B. Food may be placed in the machine's bed. The animal may be allowed to find the food inside the machine. The animal may get into the machine or be assisted to get into the machine. Once the animal is comfortable with the machine, the numbered actions as described above may be carried out for training to the device 10 in the machine.

C. The animal may be conditioned to sound as part of its placement of his or her head on the device 10 in the machine. Initially, the sound may be added away from the machine, the device 10, or the device 10 in the machine. As the animal becomes conditioned to the sound, the numbered actions described above may be carried out until the animal is comfortable with the sound and device 10, the sound and machine, and the sound with the device 10 in the machine.

D. The animal may be conditioned to one or more other distractions in the same manner as with sound.

The training of an animal with respect to the device 10 in a machine does not necessarily have to include commands. A handler may choose to use a command such as “cup” when the animal places his or her head on the device 10.

The handler may choose to use a marker word when training and/or working with an animal and the device 10 (whether or not in the machine). A marker word is a word that is used to mark a specific behavior. Use of the marker word by the handler means that the animal receives a reward. The reward should be given by the handler every time the handler uses the marker word and vice versa. The marker word may become a release command in order to inform the animal that he or she is finished with the specific task.

During all of the above steps and actions, the handler is advised to praise the animal and keep the animal motivated.

### An Exemplary Method of the Invention

The invention contemplates a method with one or more actions (some may be optional) with respect to the training of an animal to position his or her head on a device embodiment and to maintain that position without movement for the time it takes for a machine containing that device embodiment to carry out brain activity analysis.

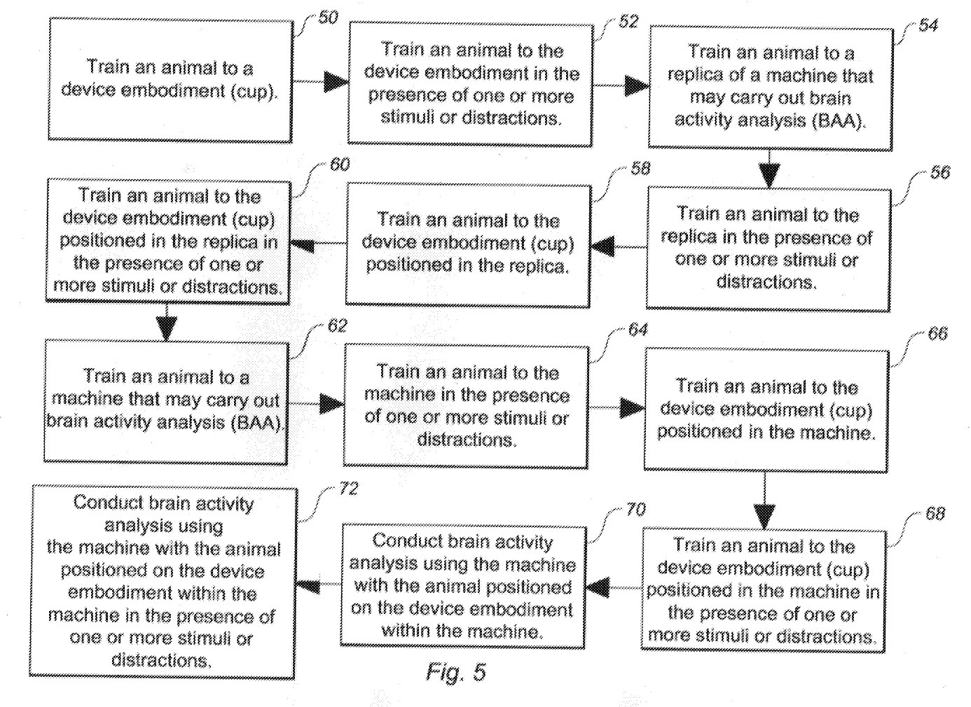

The exemplary method is presented in FIG. 5 and may first include the action of training an animal to a device embodiment (cup) in action 50. Action 52 may be optional in training the animal to the device embodiment in the presence of one or more stimuli or distractions. Another optional action is action 54 in training the animal to a replica of a machine that may carry out brain activity analysis (BAA). Optional action 56 may train the animal to the replica in the presence of one or more stimuli or distractions. Further, optional action 58 may train the animal to the device embodiment (cup) positioned in the replica. Additionally, action 60 may train an animal to the device embodiment (cup) positioned in the replica in the presence of one or more stimuli or distractions.

The exemplary method includes action 62 in training the animal to a machine that may carry out brain activity analysis (BAA). Optional action 64 may allow for the training of the animal to the machine in the presence of one or more stimuli or distractions. Action 66 trains the animal to the device embodiment (cup) positioned in the machine. Optional action 68 may train the animal to the device embodiment (cup) positioned in the machine in the presence of one or more stimuli or distractions. Action 70 conducts brain activity analysis using the machine with the animal positioned on the device embodiment within the machine. Optionally, action 72 conducts brain activity analysis using the machine with the animal positioned on the device embodiment within the machine in the presence of one or more stimuli or distractions.

The actions of the exemplary method described immediately above need not take place in the order described or numbered. As noted, one or more of the actions of the exemplary method may be optional.

### CONCLUSION

The exemplary embodiments of the invention were chosen and described above in order to explain the principles of the invention and its practical applications so as to enable others skilled in the art to utilize the inventions including various embodiments and various modifications as are suited to the particular uses contemplated. The examples provided herein are not intended as limitations of the invention. Uses with other types of apparatus, devices, items, methods, and/or systems may be appropriate. Other embodiments will suggest themselves to those skilled in the art.

1. A device to aid in conducting brain activity analysis on an animal, comprising:
a headrest for receiving at least part of the head of the animal; and
a tube connected to the headrest for passing a treat to the animal through the tube so the animal is trained to position his or her head on the headrest and to maintain his or her head on the headrest for the time it takes to carry out the brain activity analysis,
whereby the animal is trained by the treat being passed to the animal through the tube to place his or her head on the headrest and to maintain the position for the time it takes to carry out the brain activity analysis so that the brain activity analysis of the animal is facilitated.

2. The device of claim 2, further comprising a neck connecting the headrest to the tube.

3. A system to aid in conducting brain activity analysis on an animal, comprising:
a device positioned within a machine that carries out brain activity analysis;
the device including a headrest for receiving at least part of the head of the animal;
the device including a tube connected to the headrest for passing a treat to the animal through the tube so the animal is trained to position his or her head on the headrest and to maintain his or her head on the headrest for the time it takes to carry out the brain activity analysis by the machine,
whereby the animal is trained by the treat being passed to the animal through the tube to place his or her head on the headrest and to maintain the position for the time it takes to carry out the brain activity analysis by the machine so that the brain activity analysis of the animal is facilitated.

4. The system of claim 4, further comprising:
a replica of the machine;
the device being positionable within the replica for training the animal to position his or her head on the device within the replica prior to training the animal to position his or head on the device within the BAA machine; and
the device being positionable within the machine after the animal is trained to position his or head on the device within the replica so that the animal is trainable to position his or her head on the device within the machine,
whereby the conduct of the BAA on the animal is facilitated.

5. A method to conduct brain activity analysis (BAA) using a machine on an the animal, comprising:
training an animal to position his or her head on a headrest of a cup positioned within the BAA machine so that the animal maintains the position of his or her head on the headrest of the cup for the time it takes for the machine to carry out the BAA on the animal; and
conducting the brain activity analysis using the machine with the animal positioned on the device embodiment within the machine.

6. The method of claim 5, further comprising:
prior to training the animal to position his or head on the headrest of the cup positioned within the BAA machine, training the animal to position his or head on the headrest of the cup before it is positioned within the BAA machine.

7. The method of claim 6, further comprising:
after the animal is trained to position his or head on the headrest of the cup before the cup is positioned within the BAA machine, and prior to training the animal to position his or head on the headrest of the cup within the BAA machine, training the animal to position his or head on the headrest of the cup positioned in a replica of the machine.

8. The method of claim 7, further comprising:
after training the animal to position his or head on the headrest of the cup positioned in the replica of the machine, training the animal to position his or head on the headrest of the cup positioned in the replica the presence of one or more stimuli or distractions.

9. The method of claim 8, further comprising:
after conducting the brain activity analysis using the machine with the animal positioned on the device embodiment within the machine, conducting the brain activity analysis using the machine with the animal positioned on the device embodiment within the machine in the presence of one or more stimuli or distractions.
